(19) United States
(12) Patent Application Publication
(10) Pub. No.: US 2012/0289832 A1
(43) Pub. Date: Nov. 15, 2012

(54) Illumination Methods And Systems For Improving Image Resolution Of Imaging Systems

(76) Inventors: Miao Zhang, Beijing (CN); Hui Hu, Seattle, WA (US)

(21) Appl. No.: 13/517,960

(22) PCT Filed: Dec. 21, 2010
(86) PCT No.: PCT/US10/61653

§ 371 Date: Jun. 20, 2012

Related U.S. Application Data
(60) Provisional application No. 61/289,327, filed on Dec. 22, 2009.

(51) Int. Cl. A61B 1/06; A61B 8/13; A61B 6/00

(52) U.S. Cl. 600/443; 600/407; 600/476

(57) ABSTRACT

Method and systems for improving resolution of imaging systems, such as a microscope or a medical ultrasonic scanner, are provided. The resolution of the microscope is improved by reducing direct illumination of unrelated regions of an object under examination. According to an aspect of the present invention, a method is provided to reduce the direct illumination of the unrelated regions in a detectable region such as a cone of light that otherwise could generate substantial noises. In another aspect of the invention, a method is provided that focuses the illumination beams such that the width of the projected beam spot is narrowed, preventing the generation of a large amount of noise. In particular, the width of the illumination beam is narrowed such that the size of the projected illumination beam is smaller than the field of view of the microscope. In another aspect of the invention, a system according to the principles of the present invention is provided, wherein the illumination beam of light is such arranged that the overlap of the path of the illumination beam of light and the detectable region is reduced.

### FIELD OF THE INVENTION

This invention relates to illumination methods and systems for improving image resolution and reducing image noises in imaging systems, and, more particularly, to illumination methods and systems for improving resolution and reducing noises of microscopes in in-vivo, high-resolution or real-time applications.

### BACKGROUND OF THE INVENTION

In an imaging system such as a microscope or a medical ultrasonic scanner, when electromagnetic wave, such as visible light or ultrasonic wave is directed on an object, the electromagnetic wave, also referred to light, will be interacted with the object. Specifically, when an object such as a tissue is illuminated by light, a chain of interactions between light and the tissue occurs. These interactions include reflection, refraction, scattering, diffusion, diffraction, etc., all of which change the light path (i.e., redirection) and other light properties (e.g., intensity, phase, and polarization). The interactions also include absorption, which causes light to be attenuated.

There are two kinds of illumination methods in imaging systems such as microscopy: one is called trans-illumination, where the illumination light illuminates the object from one side and the imaging detection system such as objective lens of microscope is positioned on the opposite side of the object to detect the light that passes through the object. The other illumination method is called reflection illumination (also called epi-illumination), where the illumination light illuminates the object from the same side of the imaging detection system such as objective lens and the imaging detection system detects light redirected (e.g., reflected or scattered) backwards by the object. The trans-illumination method, though used to study a transparent or thin specimen, is not suitable for observing structures underneath opaque thick tissues because light cannot pass through them. In such a case, the reflection illumination (epi-illumination) method becomes the only feasible option.

Microscopic study of in-vivo targets, such as micro vascular structures, underneath tissues poses a severe low-signal, high-noise challenge. The target region of the imaging system is usually at a certain depth underneath the tissue surface. As the observation depth increases, less and less illumination light can reach and interact with the target region to form the useful signals. Moreover, these useful signals are more and more likely to be further absorbed or redirected, without being detected by the imaging system. Thus, as the observation layer goes deeper underneath the tissue surface, the intensity of the useful signals detected by imaging system becomes extremely small so that they are buried by the unrelated signals, also called noises, generated outside the target region, and the resulted images are too blurry and noisy to be useful. Hence, reducing noises is desired for the improvement of image quality of an imaging system such as intravital microscopy.

Several methods have been introduced in an attempt to improve the image quality of intravital microscopy. For example, the methods of the Orthogonal Polarization Spectral (OPS) Microscope, such as disclosed in US 2008/0045817A1, use the light polarization property to filter out light having similar polarization property to the illumination light. The methods of the Dark-Field Microscope use various optics designs to filter out the light reflected directly by the surface of the object. Existing methods offer some marginal improvements, but the challenge remains. As a result, today's intravital microscope, though capable of providing a micron or sub-micron resolution when studying thin slice specimens, can only achieve a much lower resolution when studying thick tissues. For example, in human microcirculatory studies, the image quality offered by the current intravital microscope is incapable of providing information regarding the structure of capillary wall in an in-vivo study. This limits further progress in microcirculatory studies and its potential applications in clinical research and practice.

### SUMMARY OF THE INVENTION

To address the problem of high-noise in microscopes, we have realized that since the amount of light redirected by each point is proportional to the intensity of illumination light impinging, i.e., directly illuminating, upon that point, direct illumination of an unrelated region, i.e., a region outside the target region, causes a large amount of undesired light-tissue interactions in the unrelated region that only contributes to the noises. We have further realized that the noises generated in the unrelated region are more likely to be detected if they are in the detectable region of the imaging system.

In one aspect of the invention, a reflection illumination method for an imaging system is provided. The reflection illumination method comprises illuminating an object including at least one target region with at least one illumination beam; detecting light redirected by the object from a detectable region of the imaging system; wherein the at least one illumination beam is focused such that the size of a projected illumination beam spot at the target region is reduced. As a result, the noises detected by the imaging system can be reduced, and therefore improving image sharpness and the signal-to-noise ratio.

For example, the illumination beam is focused by an objective lens of the imaging system. The objective lens is used to focus both the illumination beam and detected light redirected. The projected size of the illumination beam spot at the target region may be less than a half size of the field of view of the imaging system.

In another aspect of the invention, a method for illumination in an imaging system is provided. The method comprises illuminating an object including at least one target region with at least one illumination beam; detecting light redirected by the object from a detectable region of the imaging system; wherein at least one illumination beam is aimed such that the portion of the detectable region that is under direct illumination is reduced.

In particular, the portion of the detectable region that is under direct illumination is reduced by controlling one or more of parameters including an oblique angle of the illumination beam, the displacement of the projected illumination beam spot relative to the target region, and, a size of the projected illumination beam spot relative to the target region.

For example, the illumination beam is aimed such that the projected illumination beam spot is projected off the center of the field of view of the image system. The portion of the detectable region that is under direct illumination is less than 50% of the total detectable region of the imaging system.

In another aspect of the invention, the method of reducing direct illumination of the unrelated regions in the detectable region of the imaging system and pin-point focusing illumination beams are combined to achieve the benefits of both.

In another aspect of the invention, reducing direct illumination of the unrelated regions is achieved by adjusting aiming parameters of the light beams on the object. For example, a system is provided to allow users to control the aiming and other parameters of the above illumination methods. The aiming parameters of the illumination beam include: (1) the oblique angle of the illumination beam; (2) the displacements of the projected beam spot relative to the target region and (3) the size of the projected beam spot relative to the target region. The other parameters include wavelength, intensity, phase, and polarization of the illumination beam.

In another aspect of the invention, an imaging system is provided. The imaging system comprises an illumination system including at least one illumination beam of light illuming a object including a target region; a detection system for detecting redirected light by the object from a detectable region of the imaging system; and a beam controller for directing the path of the illumination beam of light; wherein the illumination beam of light is such arranged that the overlap of the path of the illumination beam of light and the detectable region is reduced.

In particular, the beam controller is adjustable by a user by selecting one or more of illumination parameters including an oblique angle of the illumination beam, a displacement of the projected illumination beam spot relative to the target region, and a size of the projected illumination beam spot relative to the target region. The illumination system is configured to project an illumination beam spot off the center of the field of view of the imaging system. Alternatively, the illumination system is configured to project the illumination beam spot outside the field of view of the imaging system.

For example, the illumination beam of light is provided from inside a housing of the detection system of the imaging system. The illumination and detection systems share a light directing device.

In yet another aspect of the invention, a method is provide to reduce the redirected light, including reflected as well as scattered and other, from being generated by the unrelated regions in the detectable region of the imaging system. Furthermore, a system is provided to distinguish the region of the field of view from the region of the projected beam spot. Thus, the projected beam spot is operated primarily off the center of the field of view and can be adjusted by users.

Therefore, image resolution can be improved by reducing or avoiding direct illumination of unrelated regions, particularly in the detectable region of the imaging system.

The principles of the present invention can be used in the imaging systems consisting of illumination and detection systems. Examples of such a system include microscope and medical ultrasound scanning.

### BRIEF DESCRIPTION OF THE DRAWINGS

A more complete understanding of the invention may be obtained from consideration of the following description in conjunction with the drawings in which:

FIG. 1 is a schematic block diagram showing an embodiment of the microscopic system according to the principles of the invention.

FIG. 2 shows the concept of the detectable region of the imaging system, using a schematic view of the Cone Of detected Light (COL) of an exemplary microscopic system, to illustrate one principle of the invention.

FIGS. 3A and 3B show a schematic view of how the illumination beam overlaps with the detectable region in prior art intravital microscopy systems.

FIG. 4 shows a schematic view of the Off-COL Side Illumination, which is an embodiment of the present invention.

FIG. 5 shows a schematic view of the Pinpoint Illumination, which is an embodiment of the present invention. It shows a case of the Pinpoint Illumination that is called the Pinpoint Off-COL Side Illumination, which is another embodiment of the present invention.

FIG. 6 shows a schematic view of adjusting the aiming parameters of the illumination beam by users, which is an embodiment of the present invention.

FIG. 7 is a schematic illustration showing an embodiment of the present invention that implements several embodiments of the present invention shown in FIGS. 4-6.

### DETAILED DESCRIPTION OF THE INVENTION

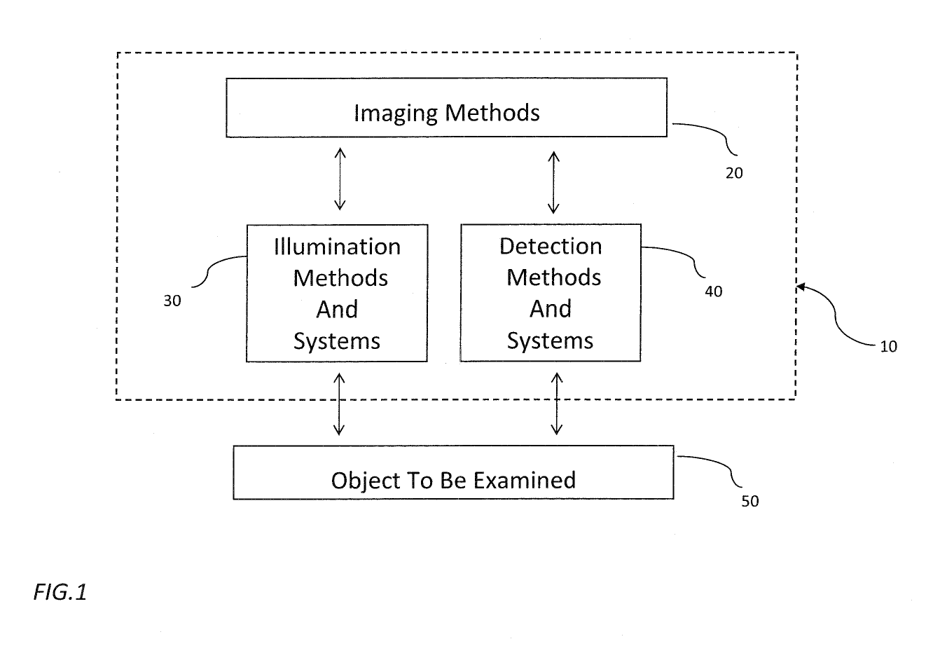

FIG. 1 shows a block diagram of an embodiment of the imaging system according to the principles of the invention. The imaging system such as a microscopic system 10 use imaging methods 20 including illumination methods and detection methods. It has an illumination system 30 to illuminate light or electromagnetic wave in general onto an object or a target 50 to be studied. The object 50, for example, can be a tissue under investigation. The object 50 include the target region of study 309. The region inside the object but outside the target region 309 is called unrelated regions of study. It also has a detection system 40 to detect redirected light from the object 50. In the case of microscope, the microscopic imaging methods 20 that can be used on this system include, but are not limited to, the optical microscope, the Fluorescence Microscope, the OPS Microscope and the Confocal Microscope.

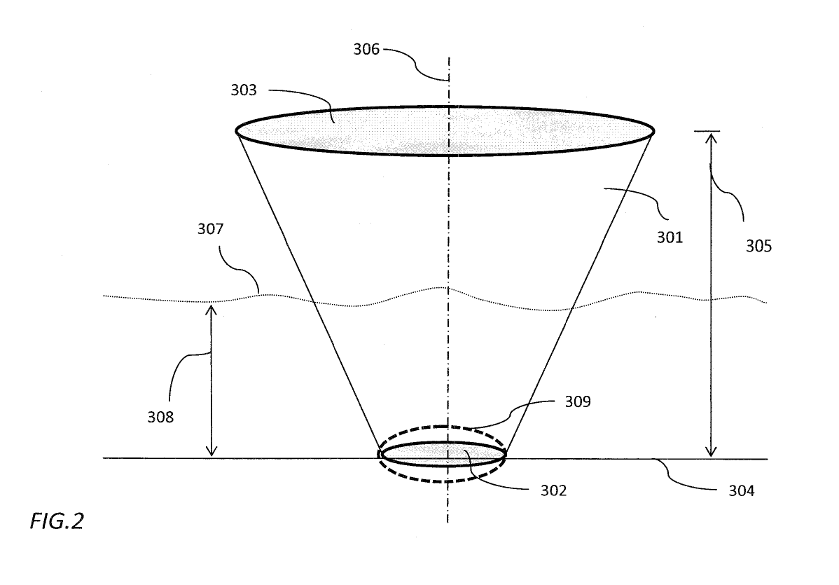

FIG. 2 shows the new concept of the detectable region of the imaging system, using an exemplary microscopic system to illustrate one principle of the invention. Referring to FIGS. 1 and 2, we introduce the detectable region 301 of the imaging system 10, such as a cone of detected light (COL) of the microscope. The Field Of View (FOV) 302 of the imaging system 10 is the cross-section of the target region 309. The imaging aperture 303 of the imaging system 10 is the opening of the imaging system 10 that determines the amount of the light to be captured in the resulted images. The detectable region of the imaging system 301 is enclosed by the FOV 302 as the apex and the imaging aperture 303 as the base. Lights redirected within the detectable region 301 are most likely to be detected by the detection system 40 of the imaging system 10. It should be understood by one of ordinary skill in the art that, the detectable region 301 can be in other forms of space such as a hollow rectangular cuboid.

Unlike the prior art microscopic system that does not consider the light-tissue interactions in its design of microscopes, we consider the light-tissue interactions and their impacts on a microscope design. The light redirected within the unrelated regions of study, if detected, only contributes as background noises. An objective of this disclosure is to design an illumination path inside the tissue relative to the detectable region 301 and FOV 302 to reduce background noises, and, therefore to increase the ratio of intensities of signals versus background noises.

It is noted that the amount of light redirected by each point in the tissue is proportional to the intensity of illumination light impinging upon that point. Thus, those points in the direct illumination paths are the primary light redirecting sources. If unrelated regions are directly illuminated, they become the primary noise-generating sources. Furthermore, if those unrelated regions right in front of the imaging detection system, i.e., inside the detectable region 301, are directly illuminated, the noises they generate are most likely to be detected by the imaging system.

An aspect of this invention is to reduce the background noises by reducing the direct illumination of unrelated regions, particularly in the detectable region 301 of the imaging system. Direct illumination of a region means the region is directly in the primary illumination beam path.

The focal plane and focal distance (also called the working distance) of the imaging system such as objective lens is denoted as 304 and 305, respectively. The axis of the imaging system such as imaging system is denoted as 306.

The detectable region 301 represents the region above the target region 309 that is directly under the imaging system. Thus, the light redirected within the detectable region 301 are most likely to be captured by the detection system 40 of the imaging system 10. The observation depth, denoted as 308, is the distance from the surface of an object, denoted as 307, to the focal plane of the imaging system 304.

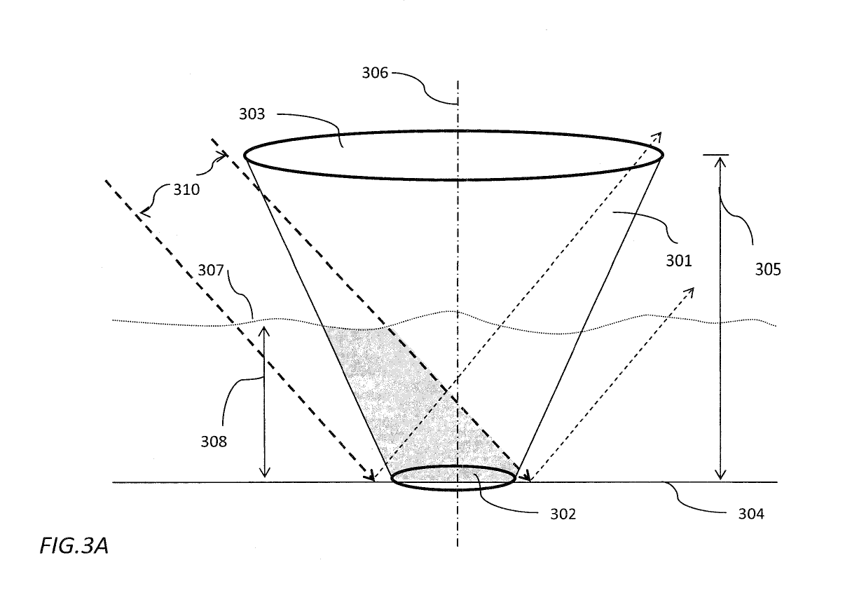

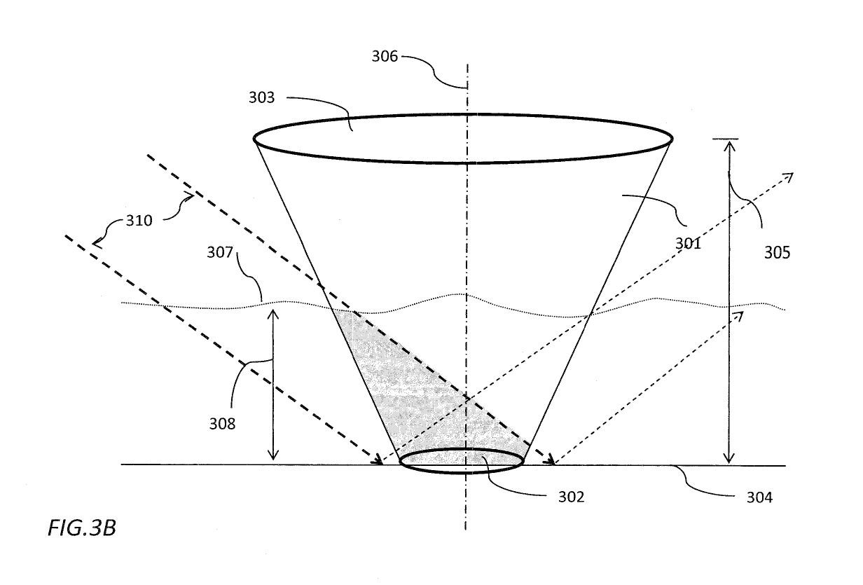

FIGS. 3A and 3B show a schematic view of how the illumination beam 310 overlaps with the detectable region 301 in prior art microscopes, specifically, A) in the Oblique Illumination; B) in the Dark Field Illumination. It is noted that the Dark Field Illumination is a special case of the Oblique Illumination where the directly reflected light are not detected by the microscope.

Lacking consideration of light-tissue interactions, prior art microscopes pay no attention to reduce the direct illumination of the unrelated regions inside the detectable region 301, which is shaded in FIGS. 3A and 3B, respectively. As a result, the prior art intravital microscope directly illuminates a substantial portion of the detectable region 301. This causes a large amount of the undesired light-tissue interactions in the region most likely to be detected by the imaging system, generating a great amount of the background noises in the resulted images that contaminate the desired signal. This is the primary reason why images in the prior art intravital microscope are unclear. The above mentioned background noises increase as the imaging aperture 303 becomes wider and the observation depth 308 becomes deeper. It increases when the focal distance 305 of the imaging system 10 decreases. We have demonstrated by an aspect of this invention that lacking a careful reduction of direct illumination of the unrelated regions, particularly in the detectable region 301, is one of the major reasons why the existing microcirculation microscopes such as capillaroscopes fail to provide acceptable results when using high magnification objective lens to observe the target deep inside an object.

As stated earlier, this disclosure, however, is to avoid or reduce the undesired light-tissue interactions that generate noise, particularly in the detectable region 301.

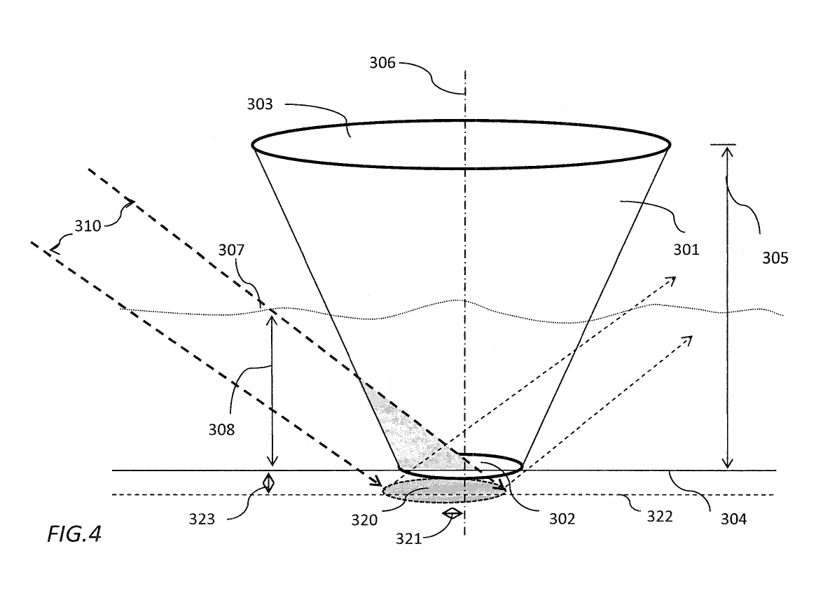

FIG. 4 shows a schematic view of the Off-COL Side Illumination, which is an embodiment of the present invention. According to the principles of the invention, the Off-COL Side Illumination carefully controls illumination beam path 310 to be outside of the detectable region or COL 301, either completely or as much as possible except nearing the target region around the FOV 302.

Referring to FIGS. 1 and 4, the illumination system 30 is arranged to project a projected beam spot 320 substantially displaced from the center of the FOV 302. The displacements can be perpendicular to and/or along the axis 306 of the imaging system, which is respectively denoted as 321 and 323. The plane parallel to the focal plane 304 that contains the focal point of the illumination beam is denoted as 322.

The Off-COL Side Illumination still directly illuminates a large amount of unrelated regions outside the detectable region or COL 301 on its way towards the target region 302. However, the noise-forming light redirected from the outside of the detectable region or COL is less likely to be detected by the imaging system such as a microscope than that from the inside.

As a result, the Off-COL Side Illumination design, compared with the prior art system shown in FIGS. 3A and 3B, can substantially reduce the direct illumination of the unrelated region inside the detectable region or COL (the shaded region), especially with the increased displacements 321, either perpendicular to and/or along the axis 306 of the imaging systems, between the center of the projected beam spot 320 and the center of the FOV 302. For example, the portion of the detectable region that is under direct illumination may be less than 25% of the total detectable region of the imaging system. Thus, the Off-COL Side Illumination can reduce the undesired light-tissue interactions that otherwise could contribute substantial background noises. It can improve the image sharpness and the signal-to-noise ratio by preventing a large amount of the background noises from generation.

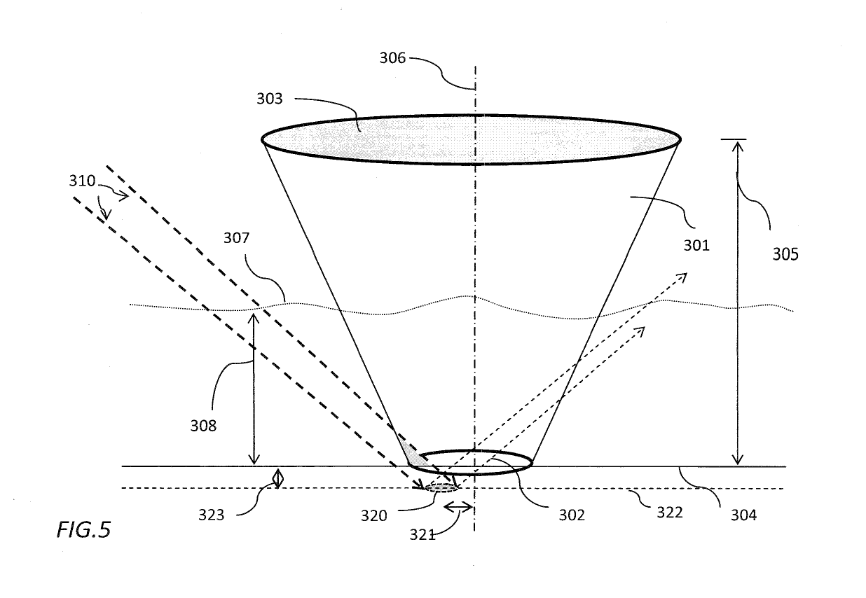

FIG. 5 shows a schematic view of the Pinpoint Illumination, in which an illumination system design is used to focus the illumination beam 310 such that the projected beam spot 320, when projected onto the focal plane of the imaging system such as objective lens 304, is substantially smaller in size than that of the FOV 302. For example, the projected size of the illumination beam spot 320 at the target region may be less than a quarter size of the FOV 302 of the imaging system. The Pinpoint Illumination can illuminate from the side (shown in FIG. 5) or from the top (not shown).

The Pinpoint Illumination, compared with the prior art shown in FIGS. 3A and 3B, substantially reduces the direct illumination of unrelated regions both inside and outside of the detectable region by using a much narrower illumination beam. It substantially reduces a large amount the undesired light-tissue interactions that otherwise could contribute substantial the background noises in the resulted images.

The Pinpoint Illumination and the Off-COL Side Illumination can be combined to achieve the benefits of both. This system and method is called the Pinpoint Off-COL Side Illumination, an example of which is shown on FIG. 5.

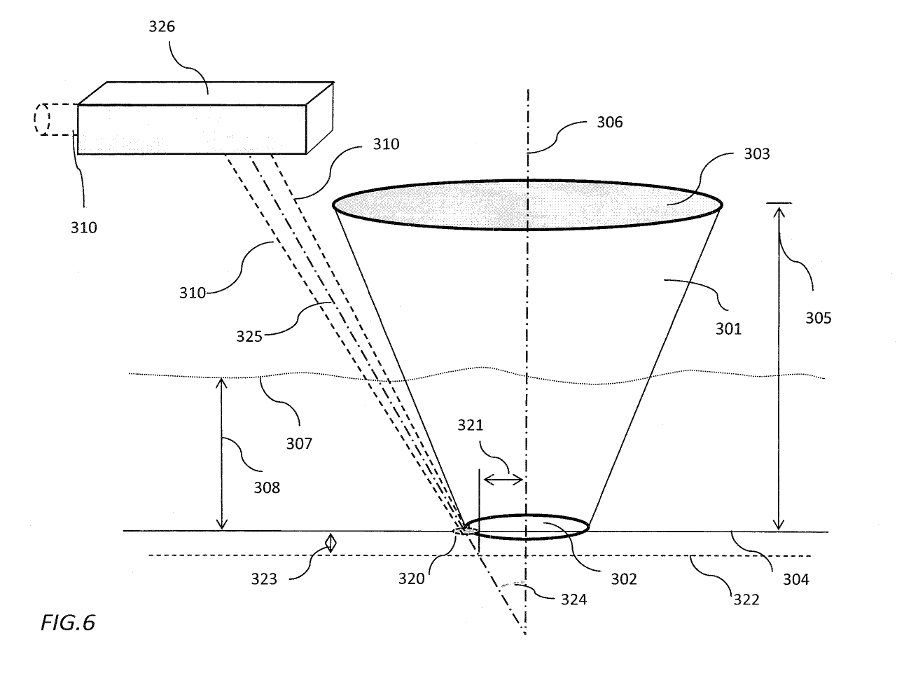

FIG. 6 discloses a beam controller system 326 according to the principles of the present invention, to allow user to control the aiming and other parameters of the above mentioned illumination systems. The aiming parameters of illumination beam 310 that can be controlled includes: 1) the oblique angle 324 of the axis 325 of the illumination beam 310, relative to the axis 306 of the imaging system such as objective lens; 2) the (center) displacements 321 and 323, of the projected beam spot 320 relative to the FOV 302 ; 3) the size of the projected beam spot relative to the FOV. The other parameters that can be controlled include wavelength, intensity, phase, and/or polarization of the illumination beam (not shown in FIG. 6). All of these parameters can be fixed in the design of the disclosed illumination systems. However, making some parameters user adjustable through the beam controller 326 allows users to achieve optimal imaging result (e.g., in term of image sharpness, or, signal-to-noise ratio) on a case by case basis, and to aim at different regions of interest.

The Off-COL Side Illumination disclosed above differs from the Oblique Illuminations used in Dark-Field Microscope in following aspects: Oblique Illumination only refers to the case in which the illumination beam 310 and the axis 306 of the imaging system are not parallel. Dark-Field Microscope only refers to the case in which the illumination beam 310 and the axis 306 of the imaging system has a large enough angle to prevent the light directly reflected near the surface layers of the object 307 from being detected. Prior art methods say nothing about reducing the direct illumination within the detectable region 301 of the imaging system. Furthermore, prior art does not distinguish the region of FOV 320 from the region of the projected beam spot 320 and typically designs these two regions to be fixed and co-centered. On the other hand, the Off-COL Side Illumination is designed to reduce the redirected light (including reflected as well as scattered, and other) from being generated in the unrelated region inside the detectable region 301, which includes both the surface layers 307 and underneath. One way to achieve this reduction is to design a system to distinguish the region of FOV 302 from the region of the projected beam spot 320. Thus, in the Off-COL Side Illumination, the projected beam spot 320 is designed to operate primarily off the center of the FOV 302, by either a fixed design or a user adjustable design (FIG. 6).

It is further noted that the design goal of either the off-centered illumination (FIG. 4) or the Pinpoint Illumination (FIG. 5) is different from that of prior art (FIG. 3). The design goal of prior art microscope is to provide a centered and uniform illumination to the entire FOV so that the intensity variation in the resulted image reflects the true variation of the target region rather than the distortion due to an illumination variation. However, requiring a centered and uniform illumination calls for a wider illumination beam width and direct illumination of a large portion of unrelated regions inside the detectable region, which means a large amount of the background noises detected in intravital microscopy.

An aspect of this invention, however, considers reducing the background noises as a higher priority requirement than maintaining illumination uniformity. It is noted that as the illumination light travels deeper and deeper into the tissue, it is more and more likely to be redirected by light-tissue interactions, more and more diffused from its projected (i.e. original) beam path. Thus, the selective (i.e., non-uniform) illumination featured by the off-center and/or Pinpoint Illumination works more effectively and selectively on the unrelated regions near the tissue surface 307 than towards the target region 309 deep into the tissue. As a result, the off-center and/or Pinpoint Illumination reduces the background noises generation more effectively and more selectively, and reduces the signal generation less effectively and less selectively. Thus, the off-center and/or Pinpoint Illumination substantially improves the overall image sharpness and the signal-to-noise ratio.

As a tradeoff for the above mentioned benefits, the intensity uniformity of the images from the off-center and/or Pinpoint Illumination may be compromised. However, this compromise can be alleviated by providing the user controllable beam aiming (FIG. 6) so that user can aim at different regions of interest. In fact, the different regions in the FOV, depending on how far off the axis of the illumination beam 325, represents different tradeoffs of signal intensity and the signal-to-noise ratio. Thus, by adjusting the direction of the illumination beam 325, users can adjust this tradeoff interactively to get desired results. Furthermore, users can obtain different images by adjusting the direction of the illumination beam 325, each optimized for different considerations (e.g., signal intensity or the signal-to-noise ratio) respectively.

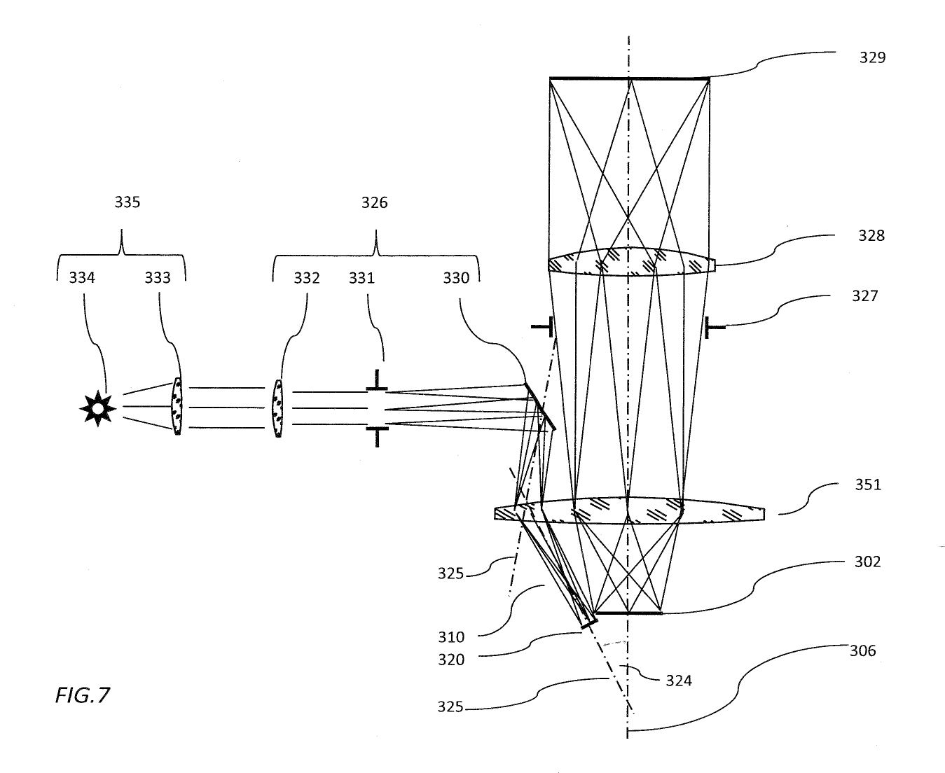

FIG. 7 discloses an embodiment of optics design that implements several embodiments of the present invention shown in FIGS. 4-6. In this embodiment, the illumination optics 30 includes: 1) a light source 335 comprising an illuminant source 334 and a light condenser 333; 2) the beam controller 326 including a focusing adjustment 332, a illuminating aperture diaphragm 331, a light path controller 330, and the outer region of the objective lens 351. The oblique angle 324 of the illumination beam can be adjusted by the light path controller 330. The projected beam spot 320 can be adjusted by the light path controller 330 that controls the displacement 321 perpendicular to the axis of the imaging system 306, and/or by the focusing adjustment 332 that controls the displacement 323 along the axis of the imaging system 306. The size of the projected beam spot 320 can be adjusted by the illuminating aperture diaphragm 331 and/or the focusing adjustment 332. The detection optics 40 include: 1) the objective lens 351, with the FOV 302 defined by it; 2) a tube lens 328; 3) optionally, an imaging aperture diaphragm 327. The objective lens 351 and the tube lens 328 combined defines the image plane 329.

This and other embodiments and operation modes of this invention are exemplified below.

Referring to FIGS. 1 and 7, the system 10 can be on tabletop or portable. It includes one or multiple Illumination Systems 30. An Illumination Systems 30 includes, but is not limited to, one or multiple illuminant sources 334, one or multiple light condensers 333, one or multiple beam controllers 326 (controlling beam aiming and other properties), and a group of lens and various filters.

Multiple illumination beams 310 can illuminate at the objects at the same or different times, using the same or different imaging methods 20, with the same or different illumination methods, with the same or different beam parameters (e.g. beam angle, position, intensity, etc). For example, two illumination beams 310 can illuminate two targets (objects), respectively, one pinpointed to a specific target (a selected region in the FOV 302), and the other providing a more uniform illumination over the entire FOV.

The illuminant source 334 of this system can use all kinds of light or wave source with any types and principles. It includes, but is not limited to, halogen lamp, mercury lamp, xenon lamp, light emitting diode, and laser diode/laser device etc. The light may be polarized or unpolarized. The light may have one or more specific wavelengths or wavelength ranges. It includes, but is not limited to, visible light, ultraviolet, or infrared light, ultrasound wave.

The illumination source 334 or 335 may be attached to a positioning device to ensure the correct position of light source. This device may include a platform that can be adjusted and translated in all directions to correctly position the light source. The positioning device may be controlled internally and/or by users.

The light condenser 333 includes, but is not limited to, one or more lenses, one or more adjustable diaphragms and various additional filters. Furthermore the Graded-Index or Gradient Index (GRIN) lens and light guide can be utilized in the light condenser 333. The purpose of the light condenser 333 is to direct the light emitted by the illuminant source 334 to the next module such as the beam controller 326. A focusing mechanism may be designed for the light condenser 333 to adjust the focal length of the illumination system.

The beam controller 326 may include, but is not limited to, one or more focusing adjustment lens 332 to adjust the focal length of the illumination system; one or more illuminating aperture diaphragm 331 to adjust the illumination beam width; and one or more light path controller 330 to change the beam direction. The light path controller 330 may include a group of prisms or flat (inclined) mirrors. The purpose of the beam controller 326 is to direct the illumination beam onto the selected region of the target region with desired illumination parameters. The beam controller 326 may have one or more controlling devices to allow one or more beam parameters (such as aiming and focusing parameters) to be controlled internally and/or by users.

The objective lens system 351 is a part of the detection systems 40 that captures the redirected light rays from the target object. The objective lens system should include, but is not limited to, a group of lenses and one or more diaphragms. The objective lens system can be designed to have a selected magnification, a selected imaging aperture and a selected working distance.

The objective lens system may also be used as a part of the illumination systems 30 (e.g., a part of the beam controller 326 or the light condenser 333) to focus the illumination beam, one embodiment of which is shown in FIG. 7.

The focusing used by the illumination beam may be either a separate lens (system) inside the same housing of the objective lens or (as shown in FIG. 7) the separate region on the same objective lens. There may be a separation device (such as walls) in the housing of the objective lens to separate the illumination from the detection and to eliminate or reduce the interference between them. In the case of sharing the same objective lens, the illumination region can use either the side region (as shown in FIG. 7) or the center region of the objective lens, while the detection region using the other region. The embodiment of providing illumination from inside the housing of the detection optical device such as objective lenses is called the internal illumination embodiment.

Alternatively, this disclosure can be implemented with a so-called external light illumination embodiment, where the illumination light is directed by a light guide external to (i.e., outside of) the objective lens of the microscope. Examples of external light guide include, but are not limited to, fiber optics, LED diode, or separate lens system.

The objective lens system may be attached with an additional anaberration system. Aberration is produced when the target is covered by extra superstratum. The anaberration system may be designed as an adjustable device, with one of the objective lens system being moved along the axial direction, in order to adapt to different situations. Alternatively, the anaberration system may also be designed as a fixed device, for example, by taking extra superstratum into account when designing the objective lens.

The objective lens may be designed as an achromatic objective, an apochromatic objective, a semi-apochromatic objective, or a plan objective etc. The objective lens can be designed as infinity conjugated or of a limited conjugated distance. The objective lens may be designed as an immersion system. The immersion medium may be oil, water, etc.

One exemplary use for the invention is the application in high-resolution human skin capillary microscopy observation. The method is adopted to reduce the background noises in the resulted images.

Using a halogen bulb as an illuminating source, the illumination light is compressed into a parallel beam that has a diameter which is much smaller than the clear aperture of an objective lens. The illumination condenser system 333 includes an aperture diaphragm and a field diaphragm which are both adjustable. The luminous flux and the beam diameter can then be controlled according to the needs of the experiment.

The light condenser system 333 guides the illumination beam to enter the beam controller 326. The beam controller 326 includes a group of optical catopters and stray light elimination diaphragms. The beam controller 326 also includes a set of precision mechanical devices. Some of the catopters can be moved and rotated by such mechanical devices. Thus, the position and angle of the illumination beam can be controlled.

An infinite conjugate distance microscope objective lens unit 351 has a design with a numerical aperture value of 0.95 and a magnification value of 20×. This objective lens unit 351 includes a diaphragm which is used to separate the regions between the illumination and the detection. The illumination beam is directed by the beam controller 326 to enter the objective lens at the illumination region of the clear aperture. There is an offset distance from the incident point to the centre axis of the objective lens. The incident axis is tilted in relation to the centre axis of the objective lens. The angle of the incident axis is adjusted by the beam controller 326.

The illumination beam can be converged by the objective lens to project onto the region near the FOV as a small spot. Adjusting the projected beam spot 320 by controlling the beam controller 326 will cause a selected target region to be illuminated. The lighting effects can be different if the projected beam spot is in a different position relative to the target.

The objective lens unit 351 has an imaging anaberration design. A method to correct this takes into account the refractive index and the thickness of the additional covering layer into an imaging formula.

Images can be observed by suitable eyepieces and they can also be recorded by a digital camera system. A software analysis may be applied to measure the velocity of flow inside blood vessels and to estimate the diameter of a capillary.

Numerous modifications and alternative embodiments of the invention will be apparent to those skilled in the art in view of the foregoing description. Accordingly, this description is to be construed as illustrative only and is for the purpose of teaching those skilled in the art the best mode of carrying out the invention. Details of the structure may be varied substantially without departing from the spirit of the invention and the exclusive use of all modifications which come within the scope of the appended claim is reserved.

What is claimed is:

1. A reflection illumination method for an imaging system comprising:
illuminating an object including at least one target region with at least one illumination beam;
detecting light redirected by the object from a detectable region of the imaging system;
wherein the at least one illumination beam is focused such that the size of a projected illumination beam spot at the target region is reduced.

2. The method of claim 1 wherein the at least one illumination beam is focused by an objective lens of the imaging system.

3. The method of claim 2 wherein the objective lens is used to focus both the illumination beam and detected light redirected.

4. The method of claim 1 wherein the projected size of the illumination beam spot at the target region is less than a half size of the field of view of the imaging system.

5. The method of claim 4 wherein the projected size of the illumination beam spot at the target region is less than a quarter size of the field of view of the imaging system.

6. A method for illumination in an imaging system comprising:
illuminating an object including at least one target region with at least one illumination beam;
detecting light redirected by the object from a detectable region of the imaging system;
wherein at least one illumination beam is aimed such that the portion of the detectable region that is under direct illumination is reduced.

7. The method of claim 6 wherein the portion of the detectable region that is under direct illumination is reduced by controlling one or more of parameters including an oblique angle of the illumination beam, the displacement of the projected illumination beam spot relative to the target region, and, a size of the projected illumination beam spot relative to the target region.

8. The method of claim 6 wherein the illumination beam is aimed such that the projected illumination beam spot is projected outside the field of view of the image system.

9. The method of claim 6 wherein the portion of the detectable region that is under direct illumination is less than 50% of the total detectable region of the imaging system.

10. The method of claim 9 wherein the portion of the detectable region that is under direct illumination is less than 25% of the total detectable region of the imaging system.

11. An imaging system comprising:
an illumination system including at least one illumination beam of light illuming a object including a target region;
a detection system for detecting redirected light by the object from a detectable region of the imaging system; and
a beam controller for directing the path of the illumination beam of light;
wherein the illumination beam of light is such arranged that the overlap of the path of the illumination beam of light and the detectable region is reduced.

12. The imaging system of claim 11 wherein the beam controller is adjustable by a user by selecting one or more of illumination parameters including an oblique angle of the illumination beam, a displacement of the projected illumination beam spot relative to the target region, and a size of the projected illumination beam spot relative to the target region.

13. The imaging system of claim 11 wherein the illumination beam of light is provided from inside a housing of the detection system of the imaging system.

14. The imaging system of claim 11 wherein illumination and detection systems share a light directing device.

15. The imaging system of claim 11 wherein the illumination is provided from inside the housing of an objective lens of the imaging system.

16. The imaging system of claim 15 wherein illumination and detection systems share the same objective lens.

17. The imaging system of claim 11 wherein the illumination system can be controlled by a user using a parameter selected from the group consisting of a wavelength of the illumination beam, an intensity of the illumination beam, a phase of the illumination beam and a polarization of the illumination beam.

18. The imaging system of claim 11 wherein the illumination system is configured to project an illumination beam spot off the center of the field of view of the imaging system.

19. The imaging system of claim 18 wherein the illumination system is configured to project the illumination beam spot outside the field of view of the imaging system.

20. The imaging system of claim 11 wherein the illumination light is visible light.

21. The imaging system of claim 11 wherein the illumination light is ultrasound wave.

22. The imaging system of claim 11 wherein the imaging system is microscopy.
